(19) United States
(12) Patent Application Publication
(10) Pub. No.: US 2024/0238591 A1
(43) Pub. Date: Jul. 18, 2024

(54) Systems and Methods to Guide Spinal Cord Stimulation Using Evoked Potentials

(71) Applicant: Boston Scientific Neuromodulation Corporation, Valencia, CA (US)

(72) Inventors: Tianhe Zhang, Studio City, CA (US); Rosana Esteller, Santa Clarita, CA (US); Rafael Carbunaru, Valley Village, CA (US)

(21) Appl. No.: 18/393,038

(22) Filed: Dec. 21, 2023

Related U.S. Application Data
(60) Provisional application No. 63/480,058, filed on Jan. 16, 2023.

(51) Int. Cl. A61N 1/36

(52) U.S. Cl. CPC A61N 1/36062; A61N 1/36031; A61N 1/36132; A61N 1/36139

(57) ABSTRACT

Methods and systems for spinal cord stimulation (SCS) are disclosed. The methods and systems involve using electrode leads implanted within the patient's spinal column to record neural responses evoked by the stimulation. The disclosed neural responses are different in several respects from electrical responses that have previously been measured in the context of SCS, such as stimulation artifacts and evoked compound action potentials (ECAPs). The disclosed neural responses typically occur later in time following the evoking stimulation pulse. Another distinguishing feature is that disclosed neural responses are generally most prominently observed with consistent, relatively unchanging amplitudes when the evoking stimulation frequency is ultra-low, for example, about 10 Hz or less. The disclosed methods and systems may use these neural responses as indications of pain, therapy, and/or another clinically relevant dimension, to direct/confirm stimulation placement, and for feedback control of stimulation parameters.

### CROSS REFERENCE TO RELATED APPLICATIONS

This is a non-provisional application of U.S. Provisional Patent Application Ser. No. 63/480,058, filed Jan. 16, 2023, which is incorporated herein by reference in its entirety, and to which priority is claimed.

### FIELD OF THE INVENTION

This application relates to Implantable Medical Devices (IMDs), and more specifically sensing signals in an implantable stimulator device.

### INTRODUCTION

Implantable neurostimulator devices are implantable medical devices (IMDs) that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability with any implantable neurostimulator device system.

An SCS system typically includes an Implantable Pulse Generator (IPG) 10 shown in FIG. 1. The IPG 10 includes a biocompatible device case 12 that holds the circuitry and a battery 14 for providing power for the IPG to function. The IPG 10 is coupled to tissue-stimulating electrodes 16 via one or more electrode leads that form an electrode array 17. For example, one or more percutaneous leads 15 can be used having ring-shaped or split-ring electrodes 16 carried on a flexible body 18. In another example, a paddle lead 19 provides electrodes 16 positioned on one of its generally flat surfaces. Lead wires 20 within the leads are coupled to the electrodes 16 and to proximal contacts 21 insertable into lead connectors 22 fixed in a header 23 on the IPG 10, which header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12.

In the illustrated IPG 10, there are thirty-two electrodes (E1-E32), split between four percutaneous leads 15, or contained on a single paddle lead 19, and thus the header 23 may include a 2×2 array of eight-electrode lead connectors 22. However, the type and number of leads, and the number of electrodes, in an IPG is application-specific and therefore can vary. The conductive case 12 can also comprise an electrode (Ec). In a SCS application, the electrode lead(s) are typically implanted in the spinal column proximate to the dura in a patient's spinal cord, preferably spanning left and right of the patient's spinal column. The proximal contacts 21 are tunneled through the patient's tissue to a distant location such as the buttocks where the IPG case 12 is implanted, at which point they are coupled to the lead connectors 22. In other IPG examples designed for implantation directly at a site requiring stimulation, the IPG can be lead-less, having electrodes 16 instead appearing on the body of the IPG 10 for contacting the patient's tissue. The IPG lead(s) can be integrated with and permanently connected to the IPG 10 in other solutions. The goal of SCS therapy is to provide electrical stimulation from the electrodes 16 to alleviate a patient's symptoms, such as chronic back pain.

IPG 10 can include an antenna 27a allowing it to communicate bi-directionally with a number of external devices used to program or monitor the IPG, such as a hand-held patient controller or a clinician's programmer (CP), as described for example in U.S. Patent Application Publication 2019/0175915. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Zigbee, MICS, and the like.

Stimulation in IPG 10 is typically provided by pulses each of which may include a number of phases such as 30a and 30b, as shown in the example of FIG. 2A. Stimulation parameters typically include amplitude (current I, although a voltage amplitude V can also be used); frequency (F); pulse width (PW) of the pulses or of its individual phases; the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E4 has been selected as an anode (during its first phase 30a), and thus provides pulses which source a positive current of amplitude +I to the tissue. Electrode E5 has been selected as a cathode (again during first phase 30a), and thus provides pulses which sink a corresponding negative current of amplitude −I from the tissue. This is an example of bipolar stimulation, in which only two lead-based electrodes are used to provide stimulation to the tissue (one anode, one cathode). However, more than one electrode may be selected to act as an anode at a given time, and more than one electrode may be selected to act as a cathode at a given time. The case electrode Ec (12) can also be selected as an electrode, or current return, in what is known as monopolar situation.

IPG 10 as mentioned includes stimulation circuitry 28 to form prescribed stimulation at a patient's tissue. FIG. 3 shows an example of stimulation circuitry 28, which includes one or more current source circuits 40ᵢ and one or more current sink circuits 42ᵢ. The sources and sinks 40ᵢ and 42ᵢ can comprise Digital-to-Analog converters (DACs), and may be referred to as PDACs 40ᵢ and NDACs 42ᵢ in accordance with the Positive (sourced, anodic) and Negative (sunk, cathodic) currents they respectively issue. In the example shown, a NDAC/PDAC 40ᵢ/42ᵢ pair is dedicated (hardwired) to a particular electrode node ei 39. Each electrode node ei 39 is connected to an electrode Ei 16 via a DC-blocking capacitor Ci 38, for the reasons explained below. The stimulation circuitry 28 in this example also supports selection of the conductive case 12 as an electrode (Ec 12), which case electrode is typically selected for monopolar stimulation. PDACs 40ᵢ and NDACs 42ᵢ can also comprise voltage sources.

Proper control of the PDACs 40ᵢ and NDACs 42ᵢ allows any of the electrodes 16 to act as anodes or cathodes to create a current through a patient's tissue, R, hopefully with good therapeutic effect. In the example shown (FIG. 2A), and during the first phase 30a in which electrodes E4 and E5 are selected as an anode and cathode respectively, PDAC 40₄ and NDAC 42₅ are activated and digitally programmed to produce the desired current, I, with the correct timing (e.g., in accordance with the prescribed frequency F and pulse width PWa). During the second phase 30b (PWb), PDAC 405 and NDAC 424 would be activated to reverse the polarity of the current. More than one anode electrode and more than one cathode electrode may be selected at one time, and thus current can flow through the tissue R between two or more of the electrodes 16.

Power for the stimulation circuitry 28 is provided by a compliance voltage VH. As described in further detail in U.S. Patent Application Publication 2013/0289665, the compliance voltage VH can be produced by a compliance voltage generator 29, which can comprise a circuit used to boost the battery 14's voltage (Vbat) to a voltage VH sufficient to drive the prescribed current I through the tissue R. The compliance voltage generator 29 may comprise an inductor-based boost converter as described in the '665 Publication, or can comprise a capacitor-based charge pump. Because the resistance of the tissue is variable, VH may also be variable, and can be as high as 18 Volts in one example.

Other stimulation circuitries 28 can also be used in the IPG 10. In an example not shown, a switching matrix can intervene between the one or more PDACs 40ᵢ and the electrode nodes ci 39, and between the one or more NDACs 42ᵢ and the electrode nodes. Switching matrices allow one or more of the PDACs or one or more of the NDACs to be connected to one or more anode or cathode electrode nodes at a given time. Various examples of stimulation circuitries can be found in U.S. Pat. Nos. 6,181,969, 8,606,362, 8,620,436, and U.S. Patent Application Publications 2018/0071520 and 2019/0083796. Much of the stimulation circuitry 28 of FIG. 3, including the PDACs 40ᵢ and NDACs 42ᵢ, the switch matrices (if present), and the electrode nodes ci 39 can be integrated on one or more Application Specific Integrated Circuits (ASICs), as described in U.S. Patent Application Publications 2012/0095529, 2012/0092031, and 2012/0095519, which are incorporated by reference. As explained in these references, ASIC(s) may also contain other circuitry useful in the IPG 10, such as telemetry circuitry (for interfacing off chip with telemetry antennas 27a and/or 27b), the compliance voltage generator 29, various measurement circuits, etc. The stimulation circuitries described herein provide multiple independent current control (MICC) (or multiple independent voltage control) to guide the estimate of current fractionalization among multiple electrodes and estimate a total amplitude that provides a desired strength. In other words, the total anodic current can be split among two or more electrodes and/or the total cathodic current can be split among two or more electrodes, allowing the stimulation location and resulting field shapes to be adjusted. For example, a “virtual electrode” may be created at a position between two physical electrodes by fractionating current between the two electrodes. In other words, the virtual electrode is not co-located with any of the physical electrodes.

Also shown in FIG. 3 are DC-blocking capacitors Ci 38 placed in series in the electrode current paths between each of the electrode nodes ci 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Although not shown, circuitry in the IPG 10 including the stimulation circuitry 28 can also be included in an External Trial Stimulator (ETS) device which is used to mimic operation of the IPG during a trial period and prior to the IPG 10's implantation. An ETS device is typically used after the electrode array 17 has been implanted in the patient. The proximal ends of the leads in the electrode array 17 pass through an incision in the patient and are connected to the externally-worn ETS, thus allowing the ETS to provide stimulation to the patient during the trial period. Further details concerning an ETS device are described in U.S. Pat. No. 9,259,574 and U.S. Patent Application Publication 2019/0175915.

Referring again to FIG. 2A, the stimulation pulses as shown are biphasic, with each pulse at each electrode comprising a first phase 30a followed thereafter by a second phase 30b of opposite polarity. Biphasic pulses are useful to actively recover any charge that might be stored on capacitive elements in the electrode current paths, such as the DC-blocking capacitors 38, the electrode/tissue interface, or within the tissue itself. To recover all charge by the end of the second pulse phase 30b of each pulse (Vc4=Vc5=0V), the first and second phases 30a and 30b are preferably charged balanced at each electrode, with the phases comprising an equal amount of charge but of the opposite polarity. In the example shown, such charge balancing is achieved by using the same pulse width (PWa=PWb) and the same amplitude (|+I|=|−I|) for each of the pulse phases 30a and 30b. However, the pulse phases 30a and 30b may also be charged balance if the product of the amplitude and pulse widths of the two phases 30a and 30b are equal, as is known.

FIG. 3 shows that stimulation circuitry 28 can include passive recovery switches 41ᵢ, which are described further in U.S. Patent Application Publications 2018/0071527 and 2018/0140831. Passive recovery switches 41ᵢ may be attached to each of the electrode nodes 39, and are used to passively recover any charge remaining on the DC-blocking capacitors Ci 38 after issuance of the second pulse phase 30b—i.e., to recover charge without actively driving a current using the DAC circuitry. Passive charge recovery can be prudent, because non-idealities in the stimulation circuitry 28 may lead to pulse phases 30a and 30b that are not perfectly charge balanced. Passive charge recovery typically occurs during at least a portion 30c (FIG. 2A) of the quiet periods between the pulses by closing passive recovery switches 41ᵢ. As shown in FIG. 3, the other end of the switches 41ᵢ not coupled to the electrode nodes 39 are connected to a common reference voltage, which in this example comprises the voltage of the battery 14, Vbat, although another reference voltage could be used. As explained in the above-cited references, passive charge recovery tends to equilibrate the charge on the DC-blocking capacitors 38 and other capacitive elements by placing the capacitors in parallel between the reference voltage (Vbat) and the patient's tissue. Note that passive charge recovery is illustrated as small exponentially-decaying curves during 30c in FIG. 2A, which may be positive or negative depending on whether pulse phase 30a or 30b has a predominance of charge at a given electrode.

FIG. 4 shows various external devices that can wirelessly communicate data with the IPG 10 and/or the ETS 80, including a patient, hand-held external controller 45, and a clinician programmer 50. Both of devices 45 and 50 can be used to wirelessly send a stimulation program to the IPG 10 or ETS 80—that is, to program their stimulation circuitries 28 and 44 to produce pulses with a desired shape and timing described earlier. Both devices 45 and 50 may also be used to adjust one or more stimulation parameters of a stimulation program that the IPG 10 or ETS 80 is currently executing. Devices 45 and 50 may also receive information from the IPG 10 or ETS 80, such as various status information, etc.

External controller 45 can be as described in U.S. Patent Application Publication 2015/0080982 for example, and may comprise either a dedicated controller configured to work with the IPG 10. External controller 45 may also comprise a general purpose mobile electronics device such as a mobile phone which has been programmed with a Medical Device Application (MDA) allowing it to work as a wireless controller for the IPG 10 or ETS 80, as described in U.S. Patent Application Publication 2015/0231402. External controller 45 includes a user interface, including means for entering commands (e.g., buttons or icons) and a display 46. The external controller 45's user interface enables a patient to adjust stimulation parameters, although it may have limited functionality when compared to the more-powerful clinician programmer 50, described shortly.

The external controller 45 can have one or more antennas capable of communicating with the IPG 10 and ETS 80. For example, the external controller 45 can have a near-field magnetic-induction coil antenna 47a capable of wirelessly communicating with the coil antenna 27a or 42a in the IPG 10 or ETS 80. The external controller 45 can also have a far-field RF antenna 47b capable of wirelessly communicating with the RF antenna 27b or 42b in the IPG 10 or ETS 80.

The external controller 45 can also have control circuitry 48 such as a microprocessor, microcomputer, an FPGA, other digital logic structures, etc., which is capable of executing instructions in an electronic device. Control circuitry 48 can for example receive patient adjustments to stimulation parameters, and create a stimulation program to be wirelessly transmitted to the IPG 10 or ETS 80.

Clinician programmer 50 is described further in U.S. Patent Application Publication 2015/0360038, and is only briefly explained here. The clinician programmer 50 can comprise a computing device 51, such as a desktop, laptop, or notebook computer, a tablet, a mobile smart phone, a Personal Data Assistant (PDA)-type mobile computing device, etc. In FIG. 4, computing device 51 is shown as a laptop computer that includes typical computer user interface means such as a screen 52, a mouse, a keyboard, speakers, a stylus, a printer, etc., not all of which are shown for convenience. Also shown in FIG. 4 are accessory devices for the clinician programmer 50 that are usually specific to its operation as a stimulation controller, such as a communication “wand” 54, and a joystick 58, which are coupleable to suitable ports on the computing device 51, such as USB ports 59 for example.

The antenna used in the clinician programmer 50 to communicate with the IPG 10 or ETS 80 can depend on the type of antennas included in those devices. If the patient's IPG 10 or ETS 80 includes a coil antenna 27a or 82a, wand 54 can likewise include a coil antenna 56a to establish near-filed magnetic-induction communications at small distances. In this instance, the wand 54 may be affixed in close proximity to the patient, such as by placing the wand 54 in a belt or holster wearable by the patient and proximate to the patient's IPG 10 or ETS 80. If the IPG 10 or ETS 80 includes an RF antenna 27b or 82b, the wand 54, the computing device 51, or both, can likewise include an RF antenna 56b to establish communication with the IPG 10 or ETS 80 at larger distances. (Wand 54 may not be necessary in this circumstance). The clinician programmer 50 can also establish communication with other devices and networks, such as the Internet, either wirelessly or via a wired link provided at an Ethernet or network port.

To program stimulation programs or parameters for the IPG 10 or ETS 80, the clinician interfaces with a clinician programmer graphical user interface (GUI) 64 provided on the display 52 of the computing device 51. As one skilled in the art understands, the GUI 64 can be rendered by execution of clinician programmer software 66 on the computing device 51, which software may be stored in the device's non-volatile memory 68. One skilled in the art will additionally recognize that execution of the clinician programmer software 66 in the computing device 51 can be facilitated by controller circuitry 70 such as a microprocessor, microcomputer, an FPGA, other digital logic structures, etc., which is capable of executing programs in a computing device. In one example, controller circuitry 70 can include any of the i5 Core Processors, manufactured by Intel Corp. Such controller circuitry 70, in addition to executing the clinician programmer software 66 and rendering the GUI 64, can also enable communications via antennas 56a or 56b to communicate stimulation parameters chosen through the GUI 64 to the patient's IPG 10.

While GUI 64 is shown as operating in the clinician programmer 50, the user interface of the external controller 45 may provide similar functionality as the external controller 45 may have similar controller circuitry, software, etc.

### SUMMARY

Disclosed here is a method of providing electrical stimulation to a patient's spinal cord using a pulse generator (PG) connected to one or more electrode leads implanted in the patient's spinal column, each electrode lead comprising a plurality of electrodes, the method comprising: using a first one or more of the plurality of electrodes as stimulating electrodes to apply evoking stimulation to the patient's spinal cord, using a second two or more of the plurality of electrodes as recording electrodes to record neural responses evoked in the patient's spinal cord by the evoking stimulation, comparing the recorded evoked neural responses at each of the recording electrodes, and using the comparison to assess the electrode lead's placement with respect to the spinal cord. According to some embodiments, the evoking stimulation has a frequency of 10 Hz or less. According to some embodiments, evoked neural response occur at least 2 milliseconds following the evoking stimulations. According to some embodiments, the evoked neural responses arise is indicative of synaptic activity within the patient's spinal cord. According to some embodiments, comparing the recorded evoked neural responses comprises extracting one or more features of the recorded neural responses. According to some embodiments, the extracted one or more features comprises one or more of a peak amplitude, an area under the curve, and a curve length. According to some embodiments, comparing the recorded evoked neural responses comprises determining an inversion location on the lead where a polarity of the evoked neural responses switch between positive polarity and negative polarity. According to some embodiments, the method further comprises using one or more patient metrics to determine a first stimulation configuration configured to provide optimized stimulation for the patient, using the first one or more of the plurality of electrodes to apply the optimized stimulation as the evoking stimulation, and determining a first value of one or more features of the recorded evoked neural response that correlates to the first stimulation configuration. According to some embodiments, the one or more patient metrics comprise a pain level and/or pain-paresthesia overlap. According to some embodiments, the one or more patient metrics comprise a patient posture. According to some embodiments, the one or more patient metrics are determined using one or more sensors and/or input received from the patient via an external device. According to some embodiments, the one or more sensors comprise an accelerometer. According to some embodiments, the one or more sensors comprise a wearable sensor. According to some embodiments, the one or more features of the recorded evoked neural response comprises one or more of a peak amplitude, an area under the curve, a curve length and an inversion location on the lead where a polarity of the evoked neural responses switch between positive polarity to negative polarity. According to some embodiments, the method further comprises determining a change in the first value of one or more features of the recorded evoked neural response and adjusting the first stimulation configuration based on the change. According to some embodiments, the PG is an implantable pulse generator (IPG). According to some embodiments, the PG is an external trial stimulator (ITS).

Also disclosed here is a non-transitory computer readable medium comprising instructions which, when executed on a computer, configured the computer to perform a method according to any of the above embodiments.

Also disclosed here is a system for providing electrical stimulation to a patient's spinal cord using a pulse generator (PG) connected to one or more electrode leads implanted in the patient's spinal column, each electrode lead comprising a plurality of electrodes, the system comprising: control circuitry configured to: use a first one or more of the plurality of electrodes as stimulating electrodes to apply evoking stimulation to the patient's spinal cord, use a second two or more of the plurality of electrodes as recording electrodes to record neural responses evoked in the patient's spinal cord by the evoking stimulation, compare the recorded evoked neural responses at each of the recording electrodes, and use the comparison to assess the electrode lead's placement with respect to the spinal cord. According to some embodiments, the evoking stimulation has a frequency of 10 Hz or less. According to some embodiments, the evoked neural response occur at least 2 milliseconds following the evoking stimulations. According to some embodiments, the evoked neural responses arise is indicative of synaptic activity within the patient's spinal cord. According to some embodiments, comparing the recorded evoked neural responses comprises extracting one or more features of the recorded neural responses. According to some embodiments, the extracted one or more features comprises one or more of a peak amplitude, an area under the curve, and a curve length. According to some embodiments, comparing the recorded evoked neural responses comprises determining an inversion location on the lead where a polarity of the evoked neural responses switch between positive polarity and negative polarity. According to some embodiments, the control circuitry is further configured to: use one or more patient metrics to determine a first stimulation configuration configured to provide optimized stimulation for the patient, use the first one or more of the plurality of electrodes to apply the optimized stimulation as the evoking stimulation, and determine a first value of one or more features of the recorded evoked neural response that correlates to the first stimulation configuration. According to some embodiments, the one or more patient metrics comprise a pain level and/or pain-paresthesia overlap. According to some embodiments, the one or more patient metrics comprise a patient posture. According to some embodiments, the one or more patient metrics are determined using one or more sensors and/or input received from the patient via an external device. According to some embodiments, the one or more sensors comprise an accelerometer. According to some embodiments, the one or more sensors comprise a wearable sensor. According to some embodiments, one or more features of the recorded evoked neural response comprises one or more of a peak amplitude, an area under the curve, a curve length and an inversion location on the lead where a polarity of the evoked neural responses switch between positive polarity to negative polarity. According to some embodiments, the control circuitry is further configured to: a change in the first value of one or more features of the recorded evoked neural response, and adjusting the first stimulation configuration based on the change. According to some embodiments, the PG is an implantable pulse generator (IPG) configured to be implanted in the patient and wherein the control circuitry is configured within the IPG. According to some embodiments, the control circuitry is configured within an external computing system configured to transmit control commands to the PG.

### BRIEF DESCRIPTION OF THE DRAWINGS

FIG. 1 shows an Implantable Pulse Generator (IPG), in accordance with the prior art.

FIGS. 2A and 2B show an example of stimulation pulses producible by the IPG, in accordance with the prior art.

FIG. 3 shows stimulation circuitry useable in the IPG, in accordance with the prior art.

FIG. 4 shows external devices able to communicate with the IPG, in accordance with the prior art.

FIG. 5 shows an improved IPG having stimulation capability and the ability to sense an ElectroSpinoGram (ESG) signal which may include Evoked Compound Action Potentials (ECAPs) caused by the simulation.

FIG. 6 shows a stimulation artifact, ECAP, and evoked synaptic potential (ESP) each in isolation and combined in an ESG.

FIG. 7 shows an algorithm for characterizing features of ESPs and using the features for closed loop feedback control of SCS.

FIG. 8 shows stimulation for evoking and recording ESPs.

FIG. 9 shows stimulation for evoking and recording ESPs.

FIG. 10 shows a GUI displaying ESPs recorded at different positions.

FIG. 11 shows a workflow for optimizing stimulation geometry and waveforms.

FIG. 12 shows a plot relating recorded ESP behavior to stimulation location and patient metrics.

FIG. 13 shows a plot for maintaining a therapeutic window using a relation of ESP feature with stimulation location and patient metrics.

FIG. 14 shows a decision matrix relating neural response changes to probable causes and outcomes.

FIG. 15 shows logical relationships between neural response changes to probable causes and outcomes.

FIG. 16 shows a pulse program with increasing amplitudes for elucidating neural response thresholds.

FIGS. 17A and 17 B show how a pulse program with increasing frequency can be used to elucidate ESP roll-off.

FIGS. 18A and 18B show an example of relating posture changes to ESP feature changes.

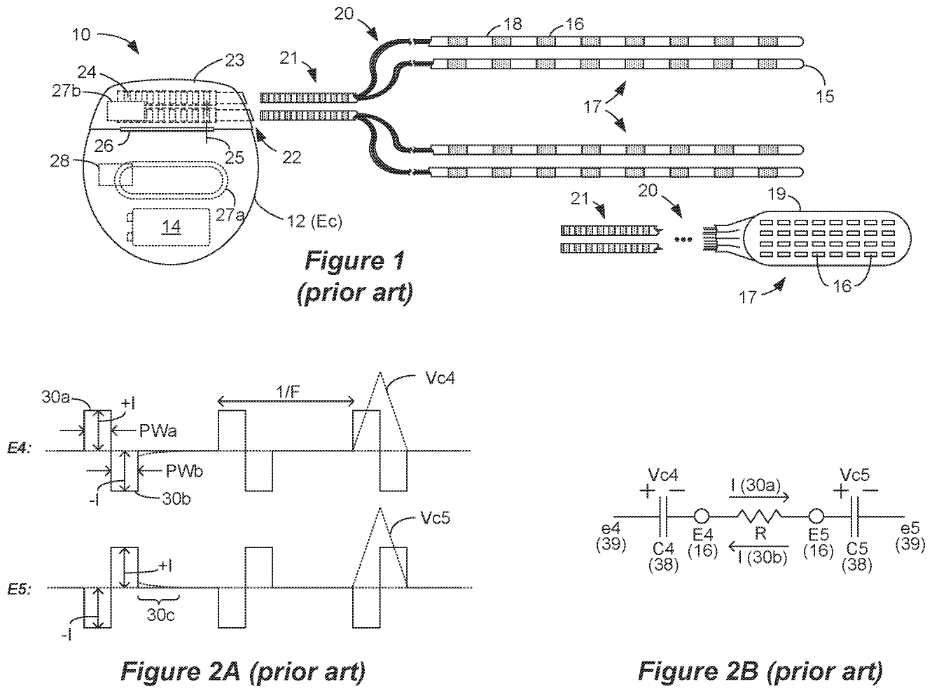

### DETAILED DESCRIPTION

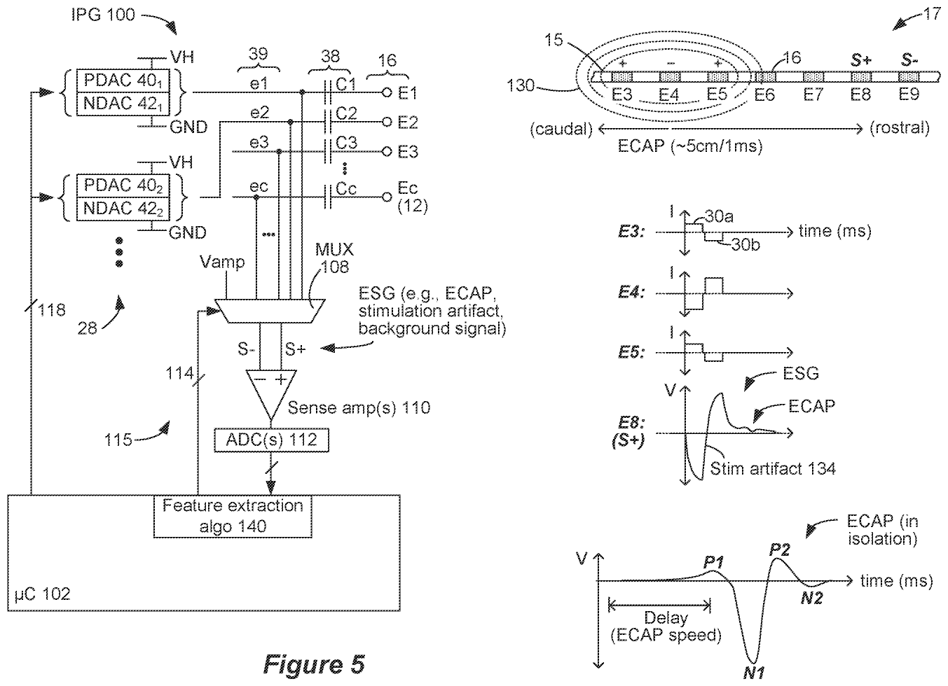

An increasingly interesting development in pulse generator systems, and in Spinal Cord Stimulator (SCS) pulse generator systems specifically, is the addition of sensing capability to complement the stimulation that such systems provide. FIG. 5 shows an IPG 100 that includes stimulation and sensing functionality. An ETS as described earlier may also include stimulation and sensing capabilities, and the circuitry shown in FIG. 5.

For example, it can be beneficial to sense a neural response in neural tissue that has received stimulation from the IPG 100. One such neural response is an Evoked Compound Action Potential (ECAP). An ECAP comprises a cumulative response provided by neural fibers that are recruited by the stimulation, and essentially comprises the sum of the action potentials of recruited neural elements (ganglia or fibers) when they “fire.” An ECAP is shown in isolation in FIG. 5, and comprises a number of peaks that are conventionally labeled with P for positive peaks and N for negative peaks, with P1 comprising a first positive peak, N1 a first negative peak, P2 a second positive peak, N2 a second negative peak, and so on. Note that not all ECAPs will have the exact shape and number of peaks as illustrated in FIG. 5, because an ECAP's shape is a function of the number and types of neural elements that are recruited and that are involved in its conduction. An ECAP is generally a small signal, and may have a peak-to-peak amplitude on the order of hundreds of microvolts or more.

FIG. 5 also shows an electrode array 17 comprising (in this example) a single percutaneous lead 15, and shows use of electrodes E3, E4 and E5 to produce pulses in a tripolar mode of stimulation, with (during the first phase 30a) E3 and E5 comprising anodes and E4 a cathode. Other electrode arrangements (e.g., bipoles, etc.) could be used as well. Such stimulation produces an electric field 130 in a volume of the patient's tissue centered around the selected electrodes. Some of the neural fibers within the electric field 130 will be recruited and fire, particularly those proximate to the cathodic electrode E4, forming ECAPs which can travel both rostrally toward the brain and caudally away from the brain. The ECAPs pass through the spinal cord by neural conduction with a speed which is dependent on the neural fibers involved in the conduction. In one example, the ECAP may move at a speed of about 5 cm/1 ms. U.S. Patent Application Publication 2020/0155019 describes a lead that can be useful in the detection of ECAPs.

ECAPs can be sensed at one or more sensing electrodes which can be selected from the electrodes 16 in the electrode array 17. Sensing preferably occurs differentially, with one electrode (e.g., S+, E8) used for sensing and another (e.g., S−, E9) used as a reference. This could also be flipped, with E8 providing the reference (S−) for sensing at electrode E9 (S+). Although not shown, the case electrode Ec (12) can also be used as a sensing reference electrode S−. Sensing reference S− could also comprise a fixed voltage provided by the IPG 100 (e.g., Vamp, discussed below), such as ground, in which case sensing would be said to be single-ended instead of differential.

The waveform appearing at sensing electrode E8 (S+) is shown in FIG. 5, which includes a stimulation artifact 134 as well as an ECAP. The stimulation artifact 134 comprises a voltage that is formed in the tissue as a result of the stimulation, i.e., as a result of the electric field 130 that the stimulation creates in the tissue. As described in U.S. Patent Application Publication 2019/0299006, the voltage in the tissue can vary between ground and the compliance voltage VH used to power the DACs, and so the stimulation artifact 134 can be on the order of Volts, and therefore significantly higher than the magnitude of stimulation-induced ECAPs. Generally speaking, the waveform sensed at the sensing electrode may be referred to as an ElectroSpinoGram (ESG) signal, which comprises the ECAP, the stimulation artifact 134, and other background signals that may be produced by neural tissue even absent stimulation. Realize that the ESG signal as shown at the sensing electrode S+ in FIG. 5 is idealized. The figures in U.S. Provisional Patent Application Publication 2022/0323764 show actual recorded ESG traces.

The magnitudes of the stimulation artifact 134 and the ECAP at the sensing electrodes S+ and S− are dependent on many factors, such as the strength of the stimulation, and the distance of sensing electrodes from the stimulation. ECAPs tend to decrease in magnitude at increasing stimulation-to-sensing distances because they disperse in the tissue. Stimulation artifacts 134 also decrease in magnitude at increasing stimulation-to-sensing distances because the electric field 130 is weaker at further distances. Note that the stimulation artifact 134 is also generally larger during the provision of the pulses, although it may still be present even after the pulse (i.e., the last phase 30b of the pulse) has ceased, due to the capacitive nature of the tissue or the capacitive nature of the driving circuitry (i.e., the DACs). As a result, the electric field 130 may not dissipate immediately upon cessation of the pulse.

It can be useful to sense in the IPG 100 features of either or both of the ECAPs or stimulation artifact 134 contained within the sensed ESG signal, because such features can be used to useful ends. For example, ECAP features can be used for feedback, such as closed-loop feedback, to adjust the stimulation the IPG 100 provides. See, e.g., U.S. Pat. No. 10,406,368; U.S. Patent Application Publications 2019/0099602, 2019/0209844, 2021/0252287, 2021/0252289, 2019/0070418, 2020/0147393 and 2022/0347479. ECAP assessment can also be used to infer the types of neural elements or fibers that are recruited, which can in turn be used to adjust the stimulation to selectively stimulate such elements. See, e.g., U.S. Patent Application Publication 2019/0275331. Assessments of ECAP features can also be used to determine cardiovascular effects, such as a patient's heart rate. See, e.g., U.S. Patent Application Publication 2019/0290900. To the extent one wishes to assess features of an ECAP that are obscured by a stimulation artifact, U.S. Patent Application Publication 2019/0366094 discloses techniques that can used to extract ECAP features from the ESG signal. As discussed in some of these references, detected ECAPs can also be dependent on a patient's posture or activity, and therefor assessment of ECAP features can be used to infer a patient's posture, which may then in turn be used to adjust the stimulation that the IPG 100 provides.

It can also be useful to detect features of stimulation artifacts 134 in their own right. For example, U.S. Patent Application Publication 2022/0323764 describes that features of stimulation artifacts can be useful to determining patient posture or activity, which again may then in turn be used to adjust the stimulation that the IPG 100 provides.

FIG. 5 shows further details of the circuitry in an IPG 100 that can provide stimulation and sensing an ElectroSpinoGram (ESG) signal. The IPG 100 includes control circuitry 102, which may comprise a microcontroller, such as Part Number MSP430, manufactured by Texas Instruments, Inc., which is described in data sheets at http://www.ti.com/microcontrollers/msp430-ultra-low-power-mcus/overview.html, which are incorporated herein by reference. Other types of controller circuitry may be used in lieu of a microcontroller as well, such as microprocessors, FPGAs, DSPs, or combinations of these, etc. Control circuitry 102 may also be formed in whole or in part in one or more Application Specific Integrated Circuits (ASICs), such as those described and incorporated earlier.

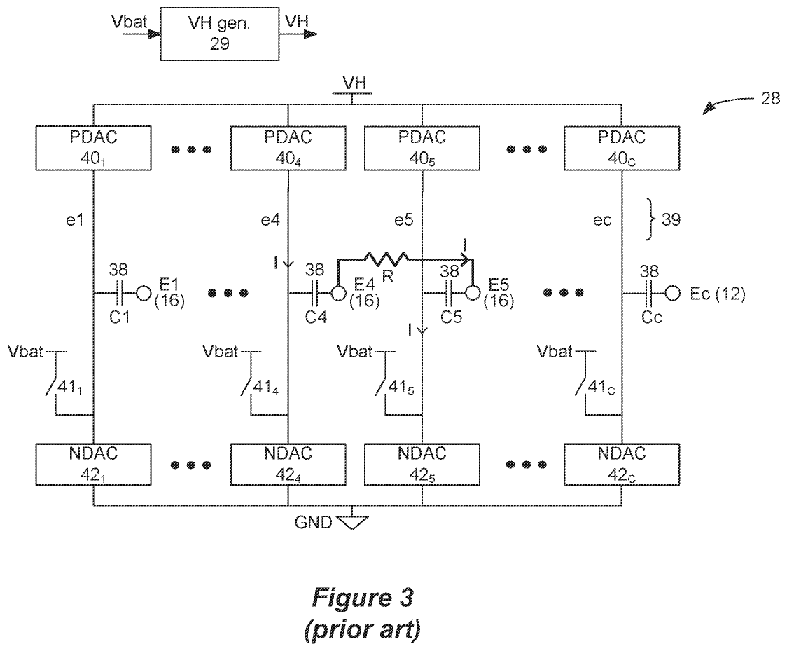

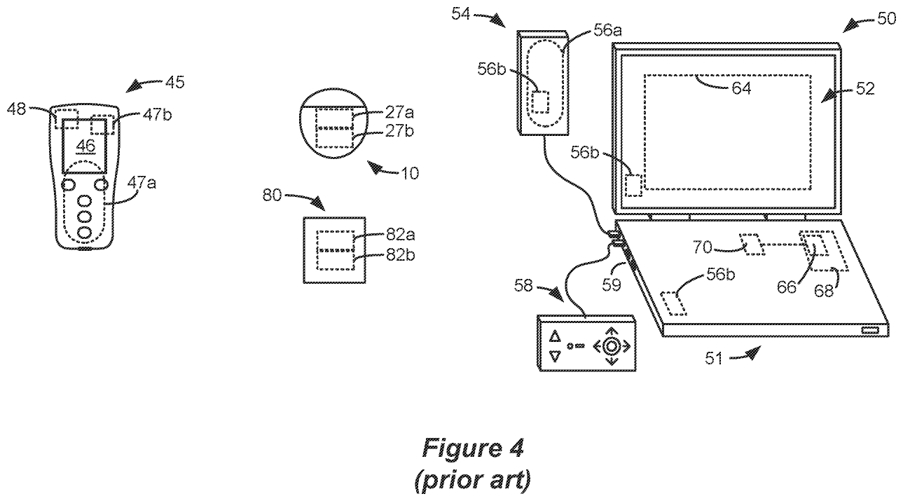

The IPG 100 also includes stimulation circuitry 28 to produce stimulation at the electrodes 16, which may comprise the stimulation circuitry 28 shown earlier (FIG. 3). A bus 118 provides digital control signals from the control circuitry 102 (and possibly from an feature extraction algorithm 140, described below) to one or more PDACs 40ᵢ or NDACs 42ᵢ to produce currents or voltages of prescribed amplitudes (I) for the stimulation pulses, and with the correct timing (PW, F) at selected electrodes. As noted earlier, the DACs can be powered between a compliance voltage VH and ground. As also noted earlier, but not shown in FIG. 4, switch matrices could intervene between the PDACs and the electrode nodes 39, and between the NDACs and the electrode nodes 39, to route their outputs to one or more of the electrodes, including the conductive case electrode 12 (Ec). Control signals for switch matrices, if present, may also be carried by bus 118. Notice that the current paths to the electrodes 16 include the DC-blocking capacitors 38 described earlier, which provide safety by preventing the inadvertent supply of DC current to an electrode and to a patient's tissue. Passive recovery switches 41ᵢ (FIG. 3) could also be present, but are not shown in FIG. 5 for simplicity.

IPG 100 also includes sensing circuitry 115, and one or more of the electrodes 16 can be used to sense signals the ESG signal. In this regard, each electrode node 39 is further coupleable to a sense amp circuit 110. Under control by bus 114, a multiplexer 108 can select one or more electrodes to operate as sensing electrodes (S+, S−) by coupling the electrode(s) to the sense amps circuit 110 at a given time, as explained further below. Although only one multiplexer 108 and sense amp circuit 110 are shown in FIG. 5, there could be more than one. For example, there can be four multiplexer 108/sense amp circuit 110 pairs each operable within one of four timing channels supported by the IPG 100 to provide stimulation. The sensed signals output by the sense amp circuitry are preferably converted to digital signals by one or more Analog-to-Digital converters (ADC(s)) 112, which may sample the output of the sense amp circuit 110 at 50 kHz for example. The ADC(s) 112 may also reside within the control circuitry 102, particularly if the control circuitry 102 has A/D inputs. Multiplexer 108 can also provide a fixed reference voltage, Vamp, to the sense amp circuit 110, as is useful in a single-ended sensing mode (i.e., to set S− to Vamp).

So as not to bypass the safety provided by the DC-blocking capacitors 38, the inputs to the sense amp circuitry 110 are preferably taken from the electrode nodes 39. However, the DC-blocking capacitors 38 will pass AC signal components (while blocking DC components), and thus AC components within the ESG signals being sensed (such as the ECAP and stimulation artifact) will still readily be sensed by the sense amp circuitry 110. In other examples, signals may be sensed directly at the electrodes 16 without passage through intervening capacitors 38.

As noted above, it is preferred to sense an ESG signal differentially, and in this regard, the sense amp circuitry 110 comprises a differential amplifier receiving the sensed signal S+ (e.g., E8) at its non-inverting input and the sensing reference S− (e.g., E9) at its inverting input. As one skilled in the art understands, the differential amplifier will subtract S− from S+ at its output, and so will cancel out any common mode voltage from both inputs. This can be useful for example when sensing ECAPs, as it may be useful to subtract the relatively large scale stimulation artifact 134 from the measurement (as much as possible) in this instance. That being said, note that differential sensing will not completely remove the stimulation artifact, because the voltages at the sensing electrodes S+ and S− will not be exactly the same. For one, each will be located at slightly different distances from the stimulation and hence will be at different locations in the electric field 130. Thus, the stimulation artifact 134 can still be sensed even when differential sensing is used. Examples of sense amp circuitry 110, and manner in which such circuitry can be used, can be found in U.S. Patent Application Publications 2019/0299006, 2020/0305744, 2020/0305745 and 2022/0233866.

The digitized ESG signal from the ADC(s) 112—inclusive of any detected ECAPs and stimulation artifacts—is received at a feature extraction algorithm 140 programmed into the IPG's control circuitry 102. The feature extraction algorithm 140 analyzes the digitized sensed signals to determine one or more ECAP features, and one or more stimulation artifact features, as described for example in U.S. Patent Application Publication 2022/0323764. Such features may generally indicate the size and shape of the relevant signals, but may also be indicative of other factors (like ECAP conduction speed). One skilled in the art will understand that the feature extraction algorithm 140 can comprise instructions that can be stored on non-transitory machine-readable media, such as magnetic, optical, or solid-state memories within the IPG 100 (e.g., stored in association with control circuitry 102).

For example, the feature extraction algorithm 140 can determine one or more neural response features (e.g., ECAP features), which may include but are not limited to:
a height of any peak (e.g., N1);
a peak-to-peak height between any two peaks (such as from N1 to P2);
a ratio of peak heights (e.g., N1/P2);
a peak width of any peak (e.g., the full-width half-maximum of N1);
an area or energy under any peak;
a total area or energy comprising the area or energy under positive peaks with the area or energy under negative peaks subtracted or added;
a length of any portion of the curve of the ECAP (e.g., the length of the curve from P1 to N2);
any time defining the duration of at least a portion of the ECAP (e.g., the time from P1 to N2);
a time delay from stimulation to issuance of the ECAP, which is indicative of the neural conduction speed of the ECAP, which can be different in different types of neural tissues;
a conduction speed (i.e., conduction velocity) of the ECAP, which can be determined by sensing the ECAP as it moves past different sensing electrodes;
a rate of variation of any of the previous features, i.e., how such features change over time;
a power (or energy) determined in a specified frequency band (e.g., delta, alpha, beta, gamma, etc.) determined in a specified time window (for example, a time window that overlaps the neural response, the stimulation artifact, etc.);
any mathematical combination or function of these variables;

Such ECAP features may be approximated by the feature extraction algorithm 140. For example, the area under the curve may comprise a sum of the absolute value of the sensed digital samples over a specified time interval. Similarly, curve length may comprise the sum of the absolute value of the difference of consecutive sensed digital samples over a specified time interval. ECAP features may also be determined within particular time intervals, which intervals may be referenced to the start of simulation, or referenced from within the ECAP signal itself (e.g., referenced to peak N1 for example).

In this disclosure, ECAP features, as described above, are also referred to as neural features or neural response features. This is because such ECAP features contain information relating to how various neural elements are excited/recruited during stimulation, and in addition, how these neural elements spontaneously fired producing spontaneous neural responses as well.

The feature extraction algorithm 140 can also determine one or more stimulation artifact features, which may be similar to the ECAP features just described, but which may also be different to account for the stimulation artifact 134's different shape. Determined stimulation artifact features may include but are not limited to:
a height of any peak;
a peak-to-peak height between any two peaks;
a ratio of peak heights;
an area or energy under any peak;
a total area or energy comprising the area or energy under positive peaks with the area or energy under negative peaks subtracted or added;
a length of any portion of the curve of the stimulation artifact;
any time defining the duration of at least a portion of the stimulation artifact;
a rate of variation of any of the previous features, i.e., how such features change over time;
a power (or energy) determined in a specified frequency band (e.g., delta, alpha, beta, gamma, etc.) determined in a specified time window (for example, a time window that overlaps the neural response, the stimulation artifact, etc.);
latency and/or time delay of any critical points in the signal, e.g. positive peaks, negative peaks, zero-crossings, etc.; and
any mathematical combination or function of these variables.

Again, such stimulation artifact features may be approximated by the feature extraction algorithm 140, and may be determined with respect to particular time intervals, which intervals may be referenced to the start or end of simulation, or referenced from within the stimulation artifact signal itself (e.g., referenced to a particular peak).

Once the feature extraction algorithm 140 determines one or more of these features, it may then be used to any useful effect in the IPG 100, and specifically may be used to adjust the stimulation that the IPG 100 provides, for example by providing new data to the stimulation circuitry 28 via bus 118. This is explained further in some of the U.S. patent documents cited above. For example, if the distance between the stimulation electrode(s) and the patient's spinal cord changes (for example, because of postural changes, coughing, movement, etc.), the stimulation may be adjusted based on the extracted features to maintain optimum therapeutic stimulation.

The SCS/neural sensing patents and applications mentioned above primarily concern ECAPs and/or stimulation artifacts. The inventors have discovered other neural responses that can be sensed, recorded, and put to useful effect during SCS. Without being bound by theory, the inventors hypothesize that the new neural responses originate from synapses and/or are evoked by synapses that connect dorsal column axons with neurons of the dorsal horn. The hypothesis of a synaptic origin of the newly observed signal is supported by the observation that CNQX (AMPA receptor antagonist that inhibits synaptic activity) causes the new neural response to disappear. Accordingly, the new neural responses are referred to in this disclosure as evoked synaptic potentials (ESPs). Since ESPs are neuronal in origin, they may be used as biomarkers for pain, therapeutic window, side effects, and/or paresthesia, as well as for directing the proper placement and control of stimulation.

ESPs differ from stimulation artifacts and ECAPs in several respects. One difference is that the ability to sense ESPs is highly dependent on the location of the sensing and stimulating electrode(s). Specifically, the ESP is most readily sensed at a location near the synapse from which it originates. The location-sensitivity of ESP sensing contrasts with sensing ECAPs, and/or stimulation artifacts, both of which propagate rostrally and caudally from their point of origin, as described above, and therefore may be sensed at various locations along the electrode lead. In other words, the ESP may be sensed at multiple locations along the spinal cord as well (if dorsal column fibers have multiple synaptic entry points into the horn), but not in the spatially continuous way that ECAPs and artifact can be sensed. Thus, ESP sensing locations remain much more constrained than those over which the ECAP may be detectable. Also, an ECAP will exhibit evidence of travel (i.e., progressive latency changes in N1 and P2) but a relatively consistent morphology. The ESP may also change morphology or even invert, depending on the location of the sensing electrode vs. the neural substrate.

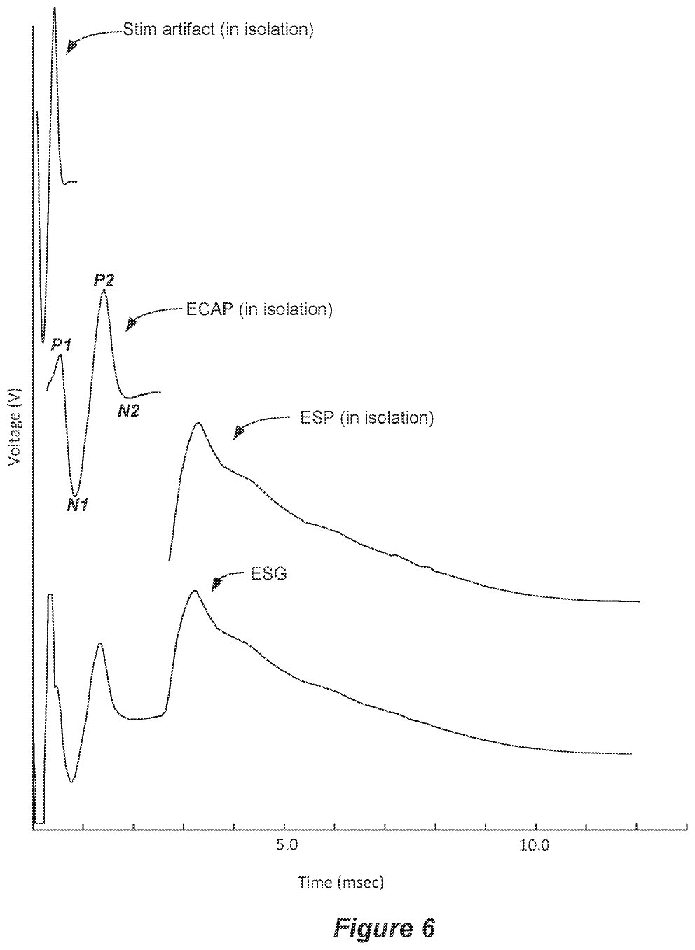

ESPs also tend to arise with a longer delay following the stimulation than ECAPs and stimulation artifacts. FIG. 6 illustrates a recorded stimulation artifact, ECAP, and ESP, each in isolation, as well as an ESG that contains each of those three recorded signals. Notice that the ESP occurs later than both the artifact and the ECAP. Accordingly, the contribution of the ESP to the ESG may be distinguished based on the time window during which the signal is recorded, as explained in more detail below.

Another distinguishing feature of ESPs is that they are generally most prominently observed with consistent, relatively unchanging amplitudes when the evoking stimulation frequency is ultra-low, for example, about 10 Hz or less. At higher frequencies, the amplitude of the ESP is significantly reduced after only a small number of periods but may still appear sporadically. In some embodiments, the ESP amplitude evoked with stimulation at 50 Hz starts decreasing after about the fourth pulse and then remain at smaller settled amplitude. With 10 Hz stimulation, the amplitude decreases more slowly, if at all. This is contrasted with ECAPs, which retain their magnitude and morphology even when SCS is applied at relatively higher frequencies (e.g., 50 Hz). Also, ESPs are also correspondingly wider than the ECAPs. ECAP width (defined by N1 to P2 width) may only be 1-2 ms, whereas ESP width, defined roughly as the width of the large positive phase may be or exceed 4-10 ms, or even larger. ESPs are generally elicited with stimulation amplitudes higher than the ECAPs but below discomfort threshold (DT). For example, some experiments have indicated that the ECAP threshold is roughly around 30% of the motor threshold while the ESP threshold is roughly around 50% to 60% of the motor threshold. This suggests, that the ECAP threshold requires the smallest stimulation amplitude, followed by the ESP threshold, which requires slightly higher amplitude. The DT and motor thresholds require even higher stimulation amplitude. These thresholds refer to the stimulation amplitude required to elicit the respective signal, such as ECAP, ESP, or motor activity.

Aspects of this disclosure relate to methods and systems for sensing, recording, characterizing, and using ESPs. For example, aspects of the disclosure involve using one or more features of the ESP as a feedback control variable for adjusting stimulation parameters, as an indication of pain, and/or determining proper placement of stimulation. For example, stimulation parameters and/or stimulation location may be adjusted to maximize a value for one or more features of the ESP. Alternatively, the parameters and/or stimulation location may be adjusted to minimize one or more features of the ESP, for example, if the ESP feature is indicative of a side effect. According to some embodiments, the ESP features may be used in conjunction with features other sensed signals, such as ECAP and/or stimulation artifact signals for feedback control.

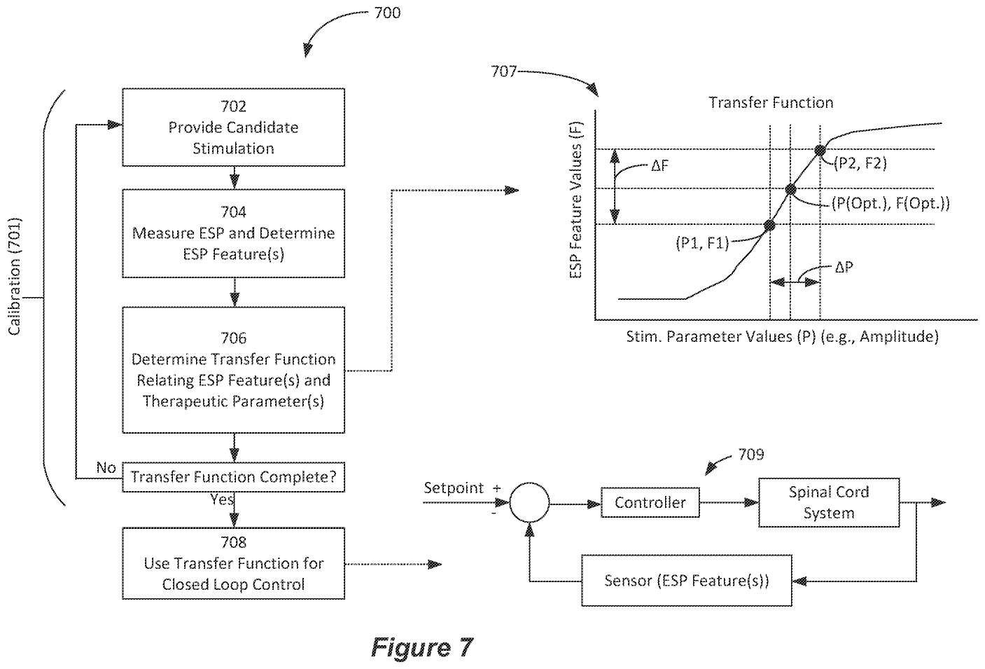

FIG. 7 illustrates an algorithm 700 for using ESPs to guide SCS. The algorithm includes calibration aspects 701 and the implementation of closed loop feedback (i.e., step 708). The calibration aspects may be performed in part or in whole using an external device to control the IPG (or ETS), such as the clinician programmer 50 and/or the external controller 45. The implementation of closed loop feedback control may be performed in the IPG, for example, using control circuitry of the IPG.

At step 702, stimulation may be provided to the patient's spinal cord using candidate stimulation parameter values. Example stimulation parameters are described above, and may include, for example, frequency, pulse width, amplitude, inter-phase interval, inter-pulse interval, and the like. The candidate stimulation parameters may also include the location of the stimulation, which may be determined based on the fractionalization of current provided to the selected active electrode contacts. For the purposes of this discussion, assume that the stimulation parameter that the algorithm 700 is seeking to optimize is stimulation amplitude. In that case, step 702 would involve providing stimulation with a first candidate amplitude.

At step 704 an ESP is measured. It should be noted, a precursor step to measuring an ESP may comprise determining an optimum location (i.e., an optimum one or more electrode contacts) at which to measure the ESP. As explained above. the ESP is location dependent, so it may be desirable to poll the various electrode contacts to find an electrode contact or contacts that best sense the ESP.

As explained above, ESPs exhibit consistent and sustained magnitude when evoked at frequencies <10 Hz but could also be observed in response to stimulation having frequencies of 50 Hz or lower. ESPs evoked at higher frequencies may exhibit amplitude decrease over subsequent stimulation periods. If the candidate therapeutic stimulation waveform has such a frequency, then ESPs evoked by the candidate therapeutic stimulation waveform may be measured. If the candidate therapeutic stimulation waveform has a higher frequency, then different stimulation waveforms may need to be applied for the purposes of evoking the ESP or the ESP that is detected according to the criteria above may attenuate over subsequent stimulation periods. In this disclosure, the term “therapeutic stimulation” means stimulation applied for a therapeutic purpose, such as treating the patient's pain. The term “evoking stimulation” applies to stimulation applied for the purpose of evoking ESPs. The evoking stimulation may have a frequency that is particularly configured to evoke ESPs, for example, below 10 Hz. The evoking stimulation may comprise a single pulse, according to some embodiments.

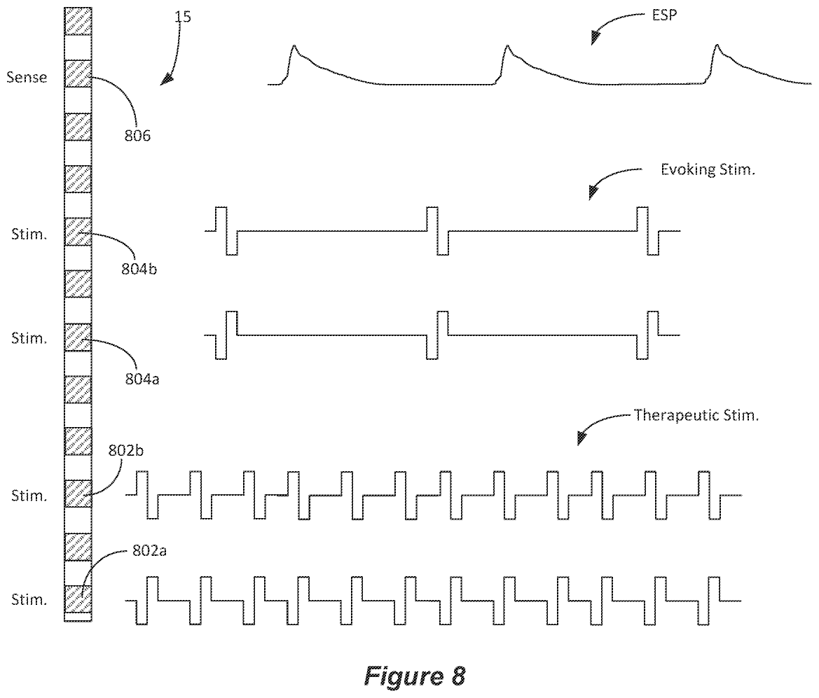

FIG. 8 illustrates an embodiment wherein therapeutic stimulation is applied at a first one or more electrode contacts (electrode contacts 802a and 802b, in the illustration) of an electrode lead 15. In the context of step 704 of the algorithm 700 (FIG. 7), the therapeutic stimulation applied at electrode contacts 802a and 802b may be candidate stimulation waveform having the candidate amplitude. Evoking stimulation may be applied simultaneously at a second one or more electrode contacts (804a and 804b, in the illustration). Note that the evoking stimulation can have a lower frequency than the therapeutic stimulation. The evoking stimulation may have an ultra-low frequency, for example, less than 10 Hz. One or more electrode contacts (electrode contact 806, in the illustration) may be used to sense the ESP. Note that the stimulation artifact and ECAP signals are omitted in FIG. 8 ESP signal, for clarity.

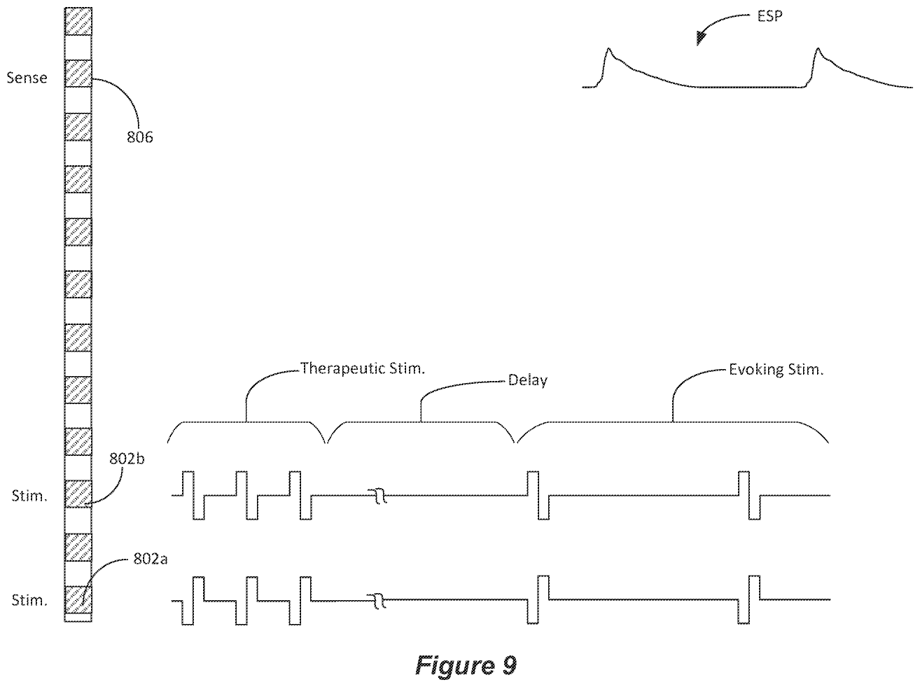

FIG. 9 illustrates another embodiment of applying both therapeutic and evoking stimulation. In FIG. 9, therapeutic stimulation is provided at a first one or more electrodes (electrode contacts 802a and 802b, in the illustration) for a first duration. A delay period is imposed for a second duration. Following the delay, evoking stimulation is issued at one or more electrodes, which may (or may not) be the same as the first one or more electrodes. The evoking stimulation is issued for a third duration, during which the ESP may be recorded at one or more recording electrodes contacts (electrode contact 806, in the illustration). The therapeutic stimulation may be resumed following the evoking stimulation, it may be delayed for a further interval.

Referring again to FIG. 7, either of the embodiments illustrated in FIG. 8 or 9 may be used to measure the ESP in response to the candidate stimulation parameters, according to step 704. Also, recall that the ESP typically arises later in time following the evoking stimulation, compared to other detectable responses, such as the stimulation artifacts and/or ECAPs, as shown in FIG. 6. Accordingly, recording the ESP (step 704) may include imposing a delay duration between issuing the evoking stimulation and the beginning of recording at the sensing electrode to avoid (as much as possible/practical) recording the stimulation artifact and the ECAP and only recording the ESP. According to some embodiments, the delay duration may be about 3 milliseconds, 2.5 milliseconds, or 2 milliseconds, etc., following the evoking stimulation, for example. According to some embodiments, the timing of the recording may be keyed to a feature of a sensed stimulation artifact or a sensed ECAP signal. For example, the algorithm may sense a feature of the ECAP, such as the N1 peak of the ECAP, and then wait an additional 1 or 1.5 milliseconds before recording the ESP. Alternatively, the algorithm may sense a feature of the stimulation artifact, such as a rising phase of the stimulation artifact, a settling phase of the artifact, etc., and time the sensing of the ESP based on the artifact feature. Also, as mentioned above, ESPs are also correspondingly wider than the ECAPs. As a consequence, some embodiments involve continuing to sense for 5-10 milliseconds or longer after the beginning of sensing, so as to capture most or all of the ESP signal. According to some embodiments, a sensed putative ESP signal may be rejected if no ECAP signal is present.

Step 704 also comprises determining one or more values for features of the ESP. The determined ESP features may be analogous to any of the features described above that may be determined for stimulation artifacts and/or ECAPs. Examples of ESP features that may be determined may be peak height, area under the curve, curve length, curve shape (such as decay rate), or any of the above-described features.

At step 706, the determined ESP feature(s) are used to develop a relationship, or “transfer function” that relates the one or more ESP features to stimulation parameters that coincide with a therapeutic goal. FIG. 7 illustrates an example of a transfer function 707, wherein ESP feature values (F) are plotted as a function of stimulation parameter values (P). In the illustrated embodiment, stimulation parameters corresponding to an optimum therapeutic outcome may be determined. Examples of therapeutic outcome may relate to pain relief, stimulation location, pain-paresthesia overlap, neural recruitment, etc. A value of a parameter (such as stimulation amplitude, pulse width, rate, total charge delivered per unit time, and/or a parameter envelope function modulation factor) that corresponds to an optimum therapeutic outcome (such as pain relief) may be denoted as P(Opt.) in Figure. For example, the stimulation value P(Opt.) may denote a stimulation amplitude that results in optimum pain relief for the patient. The range of parameter values (ΔP) between P1 and P2 may correspond to a range of stimulation amplitudes that provide acceptable pain relief. For example, the range (ΔP) may correspond to a therapeutic window. The transfer function relates the stimulation parameter values to corresponding values of the one or more ESP features. In other words, an ESP feature value F(Opt.) can be identified that corresponds to the optimum stimulation amplitude (and, accordingly, the optimum pain relief). A range of ESP feature values (ΔF) may be identified that corresponds to the range of stimulation parameter values ΔP. Steps 702-706 may be repeated until a transfer function is determined to adequate precision.

At step 708, the values of the ESP features and the transfer function may be used for closed loop feedback control of stimulation. As mentioned above, the control circuitry with in the IPG may be programmed with one or more sets of instructions configured to cause the IPG to adjust stimulation parameters based on features of the ESP. According to some embodiments, the IPG may be configured to provide therapeutic stimulation and to periodically issue evoking stimulation. The therapeutic stimulation may be sub-perception or paresthesia or a combination of both sub-perception and paresthesia. The evoking stimulation and the therapeutic stimulation may occur simultaneously, as illustrated in FIG. 8, or at different times, as illustrated in FIG. 9. If simultaneously delivered, the device may be configured to stop therapeutic stimulation afterwards for long enough such that the ESP can be sensed, for example if the therapeutic waveform was a high energy waveform. If therapeutic stimulation is being delivered at a rate that is incompatible with the ESP, the device may also be configured to pause therapeutic stimulation for 1-2 seconds before delivering the evoking stimulation.

The IPG may comprise a closed loop feedback control algorithm that is configured to use the one or more ESP feature values as control variables. Closed-loop feedback control is well known in the art and is not discussed here in detail, but the control scheme may involve controllers such PID controllers, Kalman filters, or the like. FIG. 7 illustrates a simplified control diagram 709, whereby a controller (e.g., IPG control circuitry) controls stimulation based on the ESP feature(s) sensed from the patient's spinal cord, as described above. The feedback control algorithm may adjust stimulation parameters to seek to maintain the sensed ESP features with respect to F(Opt.) or within the range ΔF, for example.

Aspects of the disclosure relate to harnessing the rostral-caudal spatial dependence of the ESP signals to useful effect. As mentioned above, prior evoked neural responses, such as ECAPS, may be detected at many locations on the spinal cord since the responses propagate through the spinal cord. ESPs, on the other hand, are spatially dependent, owing to their believed neural/synaptic origin. Both the amplitude and the shape of the ESP signal may change depending on the location of the spinal cord at which the signal is sensed. Aspects of the disclosure involve using ESP features to provide information about the electrode lead placement and for adjusting the stimulation configuration accordingly.

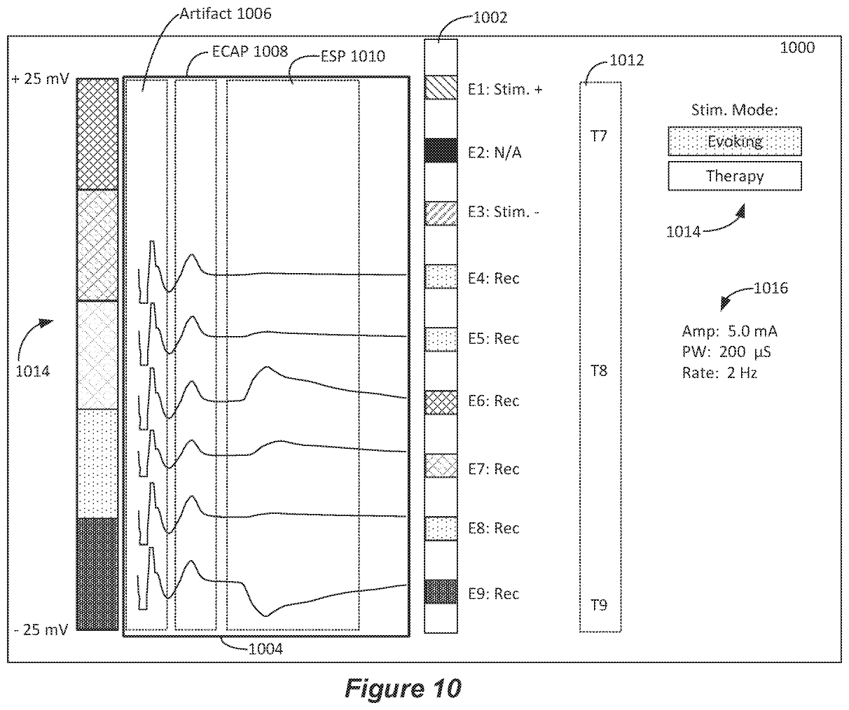

FIG. 10 illustrates an embodiment of a GUI 1000 for probing the spatial dependence of ESP signals. The GUI 1000 may be embodied on a screen of a clinician programmer 50 (FIG. 4), for example. The illustrated embodiment includes a representation of a stimulation lead 1002 having a plurality of electrodes (E1-E9). In the drawing E1 is assigned as the anode, E3 is the cathode, E2 is inactive, and E4-E9 are used as recording electrodes. The GUI may also include an indication of the vertebral level 1012 at which sensing and/or stimulation occurs. According to some embodiments, the vertebral level indication may be a representation of the patient's spinal cord.

The GUI may provide one or more ways of visualizing and/or analyzing the neural responses recorded at the recording electrodes. For example, the illustrated GUI includes a visualization panel 1004 in which the various neural response signals may be displayed. According to some embodiments, the visualization panel may be configurable to discern the different types of responses that are recorded. The illustrated visualization panel includes separate windows for the fastest responses 1006, such as stimulation artifacts, fast neural responses 1008, such as ECAPs, and slower responses 1010, such as ESPs. The neural response signals may be displayed and matched with the electrodes at which they were recorded.

The neural response signals illustrated in FIG. 10 demonstrate a property of recorded ESPs, namely, that both the ESP amplitude and the ESP polarity may vary depending on the recording location. In the illustration, very little ESP activity is recorded at electrodes E4 or E5. The electrode E6 records an ESP having a strong positive amplitude. Notice that the peak amplitude of the ESP signal decreases at E7 and is essentially absent at E8. Also notice that the ESP signal at E9 has a strong negative peak. This behavior is referred to herein as “inversion.” The illustrated ESP signals have an “inversion point,” i.e., a point at which the polarity of the ESP signal inverts, at about the location of E8. The use of inversion and inversion points will be discussed in more detail below.

The illustrated GUI also has a feature value indicator 1014 configured to display one or more feature values of the ESP signal. In the illustrated embodiment, the feature value indicator is configured to show the peak amplitude of the ESP. The feature value indicator 1214 comprises a heat map indicating peak amplitudes, ranging from −25 mV to +25 mV. Note that this voltage ranges may be adjustable, and different voltages may be used. Notice that the colors of the electrodes E4-E9 are coded based on the peak amplitude measured at the electrode. The electrodes E4 and E8 are coded with colors corresponding to near zero amplitude; electrode E6 is coded with a color corresponding to strongly positive peak amplitude; and the electrode E9 is coded with a color corresponding to a strongly negative peak amplitude. The feature value indicator may be configured to display and correlate values of other features, such as those discussed above, for example, area under the curve, curve length latency, and the like. The curves themselves may represent single epochs, or they could represent averaged, filtered, and otherwise processed epochs built up from 2 or more saved single epochs.

The GUI may also feature one or more input displays for inputting and/or adjusting stimulation and/or sensing parameters. For example, the illustrated GUI features a stimulation mode selector 1016 for switching between evoking and therapeutic stimulation and a parameter selector 1018 for inputting parameters for the applied electrical stimulation.

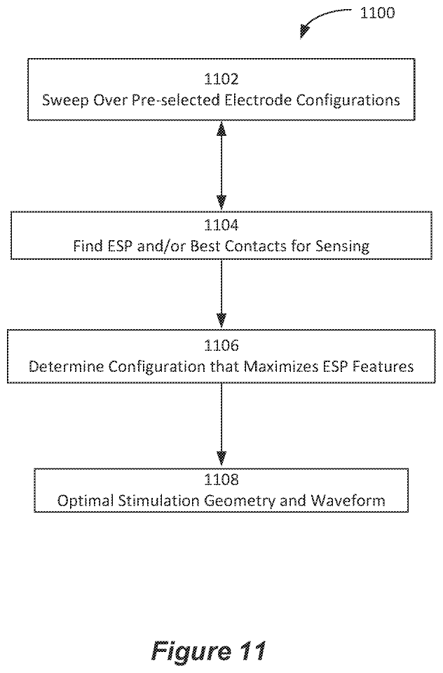

FIG. 11 illustrates an embodiments of a workflow 1100 for determining optimal electrode configurations for evoking ESP signals, for example, using a GUI 1000 (FIG. 10). At step 1102 a sweep over pre-selected electrode configurations is performed. Example electrode configurations may be bipole configurations, tripole configurations, contour bipoles, graded or continuous bipoles, wherein the stimulation current is fractioned among three or more electrodes, and the like. Essentially, step 1102 determines how stimulation current should be fractionated among the stimulating electrodes for evoking the ESP signals. This may be referred to as a “sweet spot search.” Step 1102 may also involve trying various evoking stimulation waveforms, such as those described above, for evoking the ESP signals. At step 1104, various recording electrodes may be tried to determine the best contacts for sensing the ESP signals. It should be noted that the order of steps 1104 and 1102 may be interchangeable and or the steps may be iterative. Step 1106 involves determining the ESP features that are to be observed and the configuration that maximizes those features. Any of the features mentioned above may be used, including the ESP amplitude, ACU, curve length, etc., as well as the inversion point, as described below. The output of the workflow 1100 is an optimal stimulation geometry and waveform for evoking ESP signals 1108.

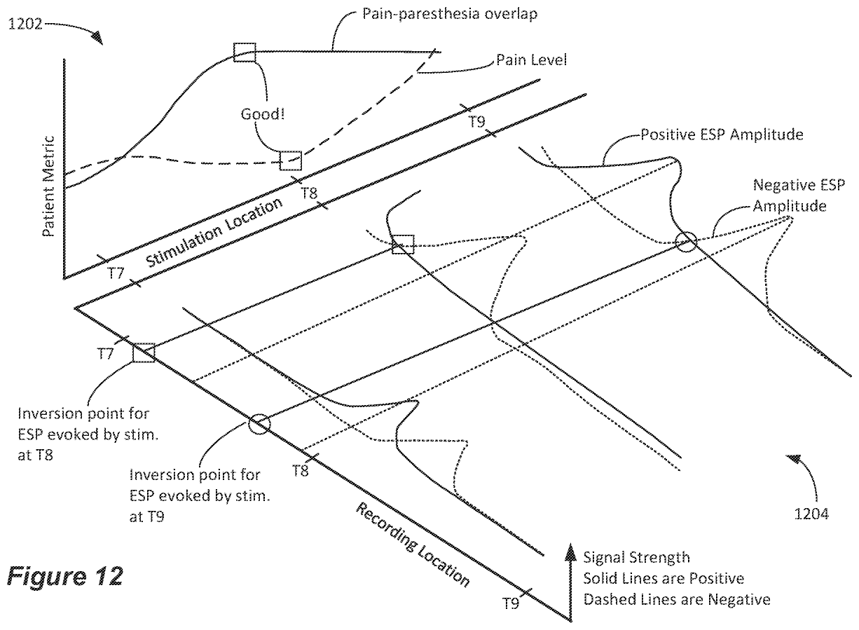

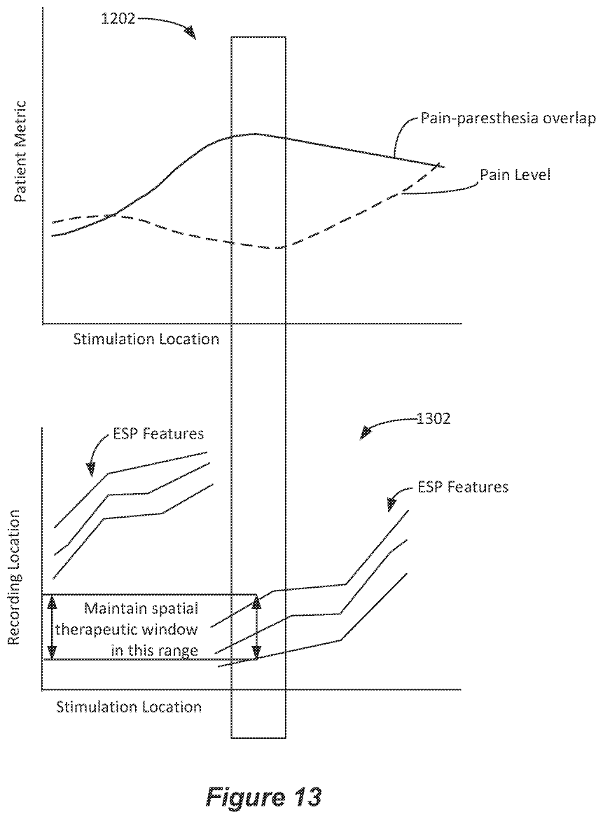

Once the optimal stimulation geometry is determined for evoking ESP signals, the ESP signals may be used for monitoring aspects of therapy. In particular, the spatial specificity of the sensed ESP signals, their polarity, and the inversion point may be used to monitor for changes in stimulation lead location/geometry and discerning such changes from other changes that might impact therapeutic outcome. FIG. 12 shows an embodiment of a three-dimensional graph correlating ESP signals with therapeutic metrics. The vertical graph 1202 shows patient metrics plotted against the vertebral location at which stimulation is provided. The patient metrics are the patient's reported pain level (dashed line) and the patient's reported pain-paresthesia overlap (solid line). The pain-paresthesia overlap indicates how well the perceived paresthesia from stimulation overlaps the dermatome at which the patient feels pain. Typically, maximum overlap is desired. Notice that when stimulation is applied near the T8 vertebral level the patient experience minimum pain and maximum pain-paresthesia overlap. That indicates that T8 is a good stimulation locations under the conditions illustrated. Likewise, stimulation near T7 or near T9 provides suboptimal results.

The horizontal plot 1204 shows ESP signals recorded at the various recording locations as a function of the stimulation location. Note that negative ESP amplitudes are drawn with dashed lines and positive ESP amplitudes are drawn with solid lines. In the illustration, when stimulation is provided at T9 the ESP shows a maximum positive amplitude that can be sensed about halfway between T7 and T8 and a maximum negative amplitude that can be sensed near T8. The ESP evoked by stimulation at T9 has an inversion point (where the ESP transitions from positive to negative polarity, marked with a circle) that can be sensed at about three quarters of the way between T7 and T8. The ESP evoked by stimulation at T8 (i.e., the optimum stimulation identified above) shows an inversion point sensed near T7 (marked with a square).

The graphs in FIG. 12 suggest that the stimulation that provides the optimum patient metric corresponds to ESP signals that have an inversion point sensed near T7, whereas less optimal stimulation results in inversion points near T8. This suggests that stimulation may be adjusted to maintain the ESP inversion point near T8 to maintain optimum therapy.

The graphs in FIG. 12 may be thought of as a clinical effects map that relates ESP features to the clinical effects experienced by the patient. It should be noted that any of the other ESP feature besides inversion points may be expressed in similar clinical effects maps. It should also be noted that other graphical representations could be used to express the clinical effects maps. Example graphical representations include cluster maps, machine learning correlations, and the like. FIG. 12 shows another embodiment of a clinical effects map. The top graph 1202 is as described above. The bottom graph 1302 shows how ESP features vary as a function of the stimulation site and recording site. The combination of 1202 and 1302 correlates those variances with the clinical effects of stimulation, providing an indication of how the ESP progresses or changes location as stimulation and/or recording is trolled along the lead. Stimulation may be maintained such that the ESP remains within an optimum spatial window, referred to herein as a spatial therapeutic window. If the ESP feature moves outside the spatial therapeutic window, then stimulation may need to be readjusted.

The ESP and its features may be used to guide stimulation, as mentioned above. The ESP may be correlated with the patient's pain state. For example, the ESP may indicate that stimulation is effective and therapeutic (positive correlation) or the ESP may correlate to the presence of a side effect (negative correlation) in some embodiments. Because of their spatial dependence, ESP signals may be used to diagnose causes of decline in therapy. Time-delay features of ESP signals may be used to determine if therapy changes are due to neural activity versus changes in the location of the lead with respect to spinal cord. Amplitude/intensity features may be used to distinguish between stimulation contact changes and sensing contact changes. If the distance change is only at the sensing contact, then changes in the ESP may not be indicative of a need to adjust stimulation. If the distance change is only at the stimulation contact, then changes in the ESP may call for adjustment. If the distance change is at both the stimulation and the sensing contact, then stimulation adjustment may be called for, but with appropriate weighting.

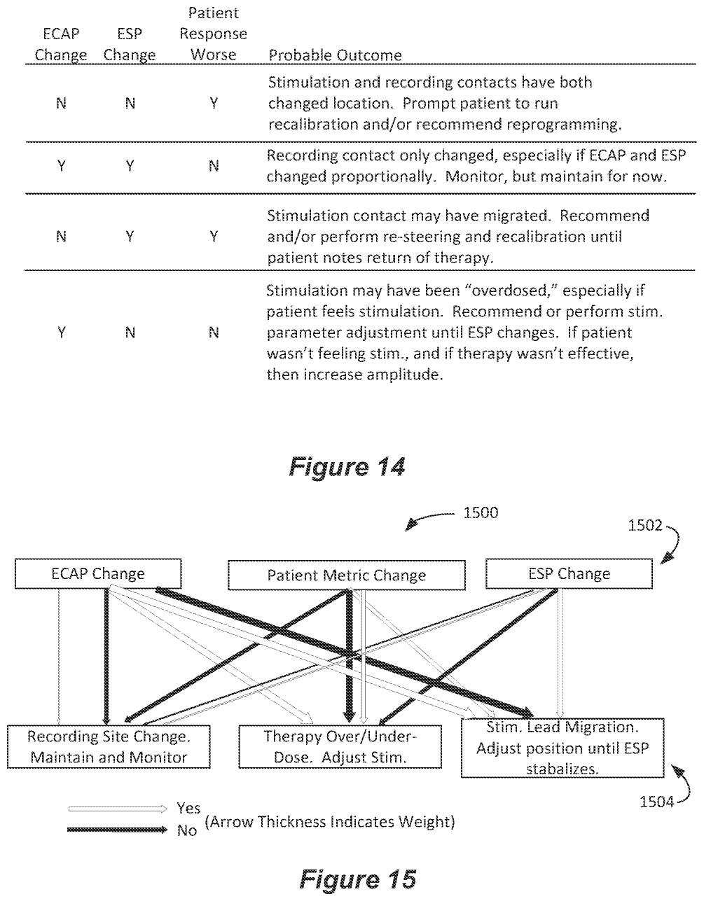

Some disclosed embodiments involve using features of both faster evoked neural responses, such as ECAPs, and the slower ESPs to diagnose changes in therapy. FIG. 14 illustrates a decision matrix correlating a patient reported decline in therapy with changes in ECAP and ESP features. The decision matrix may be used to define a control policy of how the stimulation system will respond to various indicators derived from the ECAP, ESP, and patient metric. If the patient reports a decline in therapy but neither the ECAP feature nor the ESP feature changes, then the cause may be a migration of the entire lead. In such a circumstances, the patient may be prompted to run a recalibration program to correct the stimulation configuration. A change in both the ECAP and the ESP features, but no change in the patient's therapy may indicate that only the recording lead has changed. In such circumstances the system may continue to monitor the therapy but take no further action. A change in the ECAP but no change in the ESP, accompanied by a decline in therapy, may indicate that the stimulation contact has migrated. In such circumstances a recalibration and/or adjustment of stimulation location may be needed. A change in the ECAP but no change in the ESP or in the patient response may indicate that the patient is over- or underdosed. The system may recommend a stimulation parameter adjustment.

FIG. 15 shows an embodiment of an interface 1500 for configuring a control policy for how the system responds to various indicators derived from the ECAP, ESP, and patient metrics based on the decision matrix shown in FIG. 14. Changes in the observed ECAP, ESP, and patient metrics are shown on the top (1502) and consequences are shown on the bottom (1504). The colors of the arrow indicate if a consequence is likely, with white arrows signifying “yes” and black arrows signifying “no.” The thickness of the arrows indicate a weight associated with the correlation denoted by the arrows. According to some embodiments, a user may reconfigure how the arrows are laid out and the weights of the arrows. The defined relationships may be used to encode decision trees based on combinations of changes and based on mutual logic. The indicated changes in the top row values may trigger interrupts within the control circuitry, causing the system to run predefined or user specified programs, such as recalibration programs. For example, such programs may run evoking stimulation known to evoke either ECAP and/or ESP and use the ECAP/ESP features to infer stimulation versus recording electrode changes and/or stimulation/target concordance. Various evoking stimulation waveforms are described above, and further evoking stimulation paradigms are discussed further below.

As mentioned multiple times herein, the stimulation used to evoke neural responses such as ECAPs, ESPs, and the like may be the same as the therapeutic stimulation or it may be different. According to some embodiments, the evoking stimulation may be interleaved with the therapeutic stimulation and/or it may be provided simultaneously or a different times than the therapeutic stimulation.

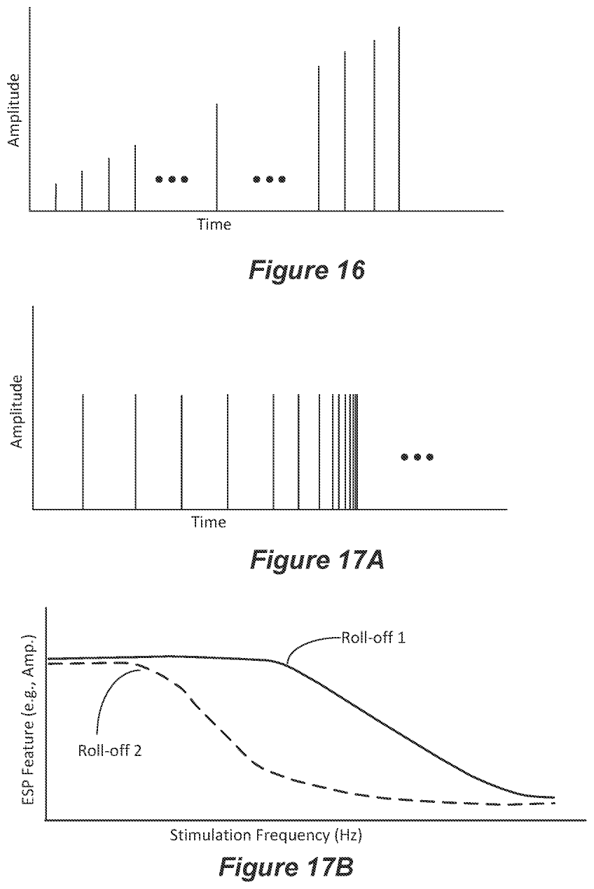

According to some embodiments, one or more stimulation parameters may be titrated to determine the optimum evoking stimulation for evoking the various neural responses. Any stimulation parameter may be used as the basis of titration and the observable may be any of the evoked response features described herein. FIG. 16 illustrates an embodiment wherein stimulation amplitude is titrated to determine the amplitude thresholds for evoking an ECAP and an ESP. Stimulation pulses or pulse packets are delivered, and the amplitudes of the pulses are increased until various evoked responses are observed. The stimulation preferably has a frequency of less than 50 Hz, and more preferably less than 10 Hz. The amplitude may begin at 0 mA or some other low value, such as a fraction of the patient's perception threshold. As the amplitude of the stimulation is increased, thresholds, such as the ECAP threshold, the patient's perception threshold (i.e., the minimum amplitude at which the patient experiences paresthesia), the ESP threshold, the patient's comfort threshold, etc., may be noted. The relationship between the ESP and ECAP thresholds may be deterministic or may be patient-specific. According to some embodiments, the ESP threshold may be predictable based on the ECAP threshold, or vise-versa, and/or the patient perception/comfort thresholds may be predictable based on one or more of the evoked neural responses. According to some embodiments, the amplitudes may be stepped down instead of up.

FIGS. 17A and 17B show an embodiment wherein the frequency of stimulation is titrated. As explained above, ESPs are evoked at low frequencies and decay at higher frequencies. Accordingly, the frequency dependency can be used to confirm that a recorded signal is indeed attributable to ESP. According to some embodiments, the morphology and/or features extracted from the morphology of the ESP can be plotted as a function of stimulation frequency. Various frequency-dependent morphological features may be tracked and changes in such features may signal a need to reprogram, recalibrate, or take other action. Example morphological features include amplitude differences, the frequency “roll-off” point, decay slop difference, final (high frequency) ESP amplitudes, or the like. Calibration curves can be checked against common/preloaded references of ESP characteristics to ensure that the signal is consistent with ESP morphology. A flag or interrupt can be thrown if the signal characteristics do not match. If there is no match, the total amplitude of the signal should also be checked. A flag/interrupt could be thrown for an amplitude above some factor of the noise floor (e.g. 125% or above), whereas if signal amplitude sinks to at or below noise floor, the signal is simply assumed not to be detectable (i.e. feature value set to 0 or the like).

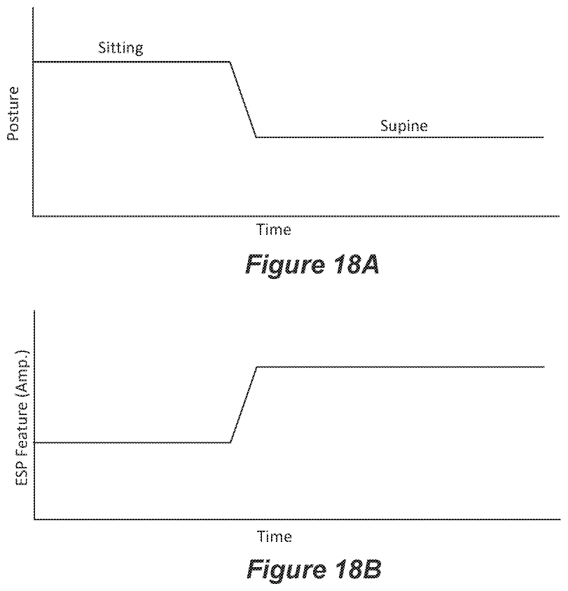

According to some embodiments, the ESP and its features may be used or correlated with patient state metrics provided by sensors, such as accelerometers (either wearable, or configured within the IPG) or other wearable or implantable sensors. The patient state metric may be anything that is shown to correlate with ESP features, such as breath cycle, heartbeat, blood pressure, posture, etc. FIGS. 18A and 18B show a correlation between the patient's posture (sitting versus supine) and an ESP feature (amplitude). The patient posture may be measured using an accelerometer, for example. Once a relationship between the patient state and the ESP feature is established, the system can intervene when the sensor detects a change that impacts the ESP feature. For example, the system may deliver evoking pulses that vary inversely with the change in the ESP feature to maintain the ESP feature within a pre-determined range.

As mentioned above, embodiments of the disclosure relate to using ESP features as an observable control variable for closed-loop feedback control for maintaining therapy. One embodiment involves using ESP as an indicator to restore or aid with balance and/or gait, for example, with Parkinson patients. The ESP may be related with proprioceptive neurons (Type I and Type II myelinated fibers), which are large diameter (e.g., larger than Aβ fibers). For Parkinson patients, ESP detection may be performed at the cervical and/or high thoracic spinal cord. Such applications may be performed similarly to the pain control modalities discussed above, whereby ESP features are correlated with severity of motor symptoms and/or accelerometry instead of (or in addition to) pain.

According to some embodiments, ESP features may correlate to a side effect. If ESP features are a sign of discomfort rather than relief, then ESP features may inform stimulation to avoid. An ESP threshold may be determined based on minimum stimulation to evoke ESP. This may involve a minimum required stimulation amplitude, minimum number of averaged epochs needed to identify/extract ESP features, etc. Stimulation may be titrated to keep the intensity at or below such a threshold (or a predetermined fraction thereof). Associated methods may involve user-configured numbers of epochs to average, ranges, signal-to-noise ratios (SNR), channels to highlight for avoidance, etc.

Although particular embodiments of the present invention have been shown and described, the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

1. A method of providing electrical stimulation to a patient's spinal cord using a pulse generator (PG) connected to one or more electrode leads implanted in the patient's spinal column, each electrode lead comprising a plurality of electrodes, the method comprising:
using a first one or more of the plurality of electrodes as stimulating electrodes to apply evoking stimulation to the patient's spinal cord,
using a second two or more of the plurality of electrodes as recording electrodes to record neural responses evoked in the patient's spinal cord by the evoking stimulation,
comparing the recorded evoked neural responses at each of the recording electrodes, and
using the comparison to assess the electrode lead's placement with respect to the spinal cord.

2. The method of claim 1, wherein the evoking stimulation has a frequency of 10 Hz or less.

3. The method of claim 1, wherein the evoked neural response occurs at least 2 milliseconds following the evoking stimulations.

4. The method of claim 1, wherein the evoked neural responses is indicative of synaptic activity within the patient's spinal cord.

5. The method of claim 1, wherein comparing the recorded evoked neural responses comprises extracting one or more features of the recorded neural responses.

6. The method of claim 5, wherein the extracted one or more features comprises one or more of a peak amplitude, an area under the curve, and a curve length.

7. The method of claim 1, wherein comparing the recorded evoked neural responses comprises determining an inversion location on the lead where a polarity of the evoked neural responses switch between positive polarity and negative polarity.

8. The method of claim 1, further comprising:
using one or more patient metrics to determine a first stimulation configuration configured to provide optimized stimulation for the patient,
using the first one or more of the plurality of electrodes to apply the optimized stimulation as the evoking stimulation, and
determining a first value of one or more features of the recorded evoked neural response that correlates to the first stimulation configuration.

9. The method of claim 8, wherein the one or more patient metrics comprise a pain level and/or pain-paresthesia overlap.

10. The method of claim 8, wherein the one or more patient metrics comprise a patient posture.

11. The method of claim 8, wherein the one or more patient metrics are determined using one or more sensors and/or input received from the patient via an external device.

12. The method of claim 11, wherein the one or more sensors comprise an accelerometer.

13. The method of claim 11, wherein the one or more sensors comprise a wearable sensor.

14. The method of claim 8, wherein one or more features of the recorded evoked neural response comprises one or more of a peak amplitude, an area under the curve, a curve length and an inversion location on the lead where a polarity of the evoked neural responses switch between positive polarity to negative polarity.

15. The method of claim 8, further comprising:
determining a change in the first value of one or more features of the recorded evoked neural response and
adjusting the first stimulation configuration based on the change.

16. The method of claim 1, wherein the PG is an implantable pulse generator (IPG).

17. The method of claim 1, wherein the PG is an external trial stimulator (ITS).

18. A system for providing electrical stimulation to a patient's spinal cord using a pulse generator (PG) connected to one or more electrode leads implanted in the patient's spinal column, each electrode lead comprising a plurality of electrodes, the system comprising:
control circuitry configured to:
use a first one or more of the plurality of electrodes as stimulating electrodes to apply evoking stimulation to the patient's spinal cord,
use a second two or more of the plurality of electrodes as recording electrodes to record neural responses evoked in the patient's spinal cord by the evoking stimulation,
compare the recorded evoked neural responses at each of the recording electrodes, and
use the comparison to assess the electrode lead's placement with respect to the spinal cord.

19. The system of claim 18, wherein the evoking stimulation has a frequency of 10 Hz or less and the evoked neural response occurs at least 2 milliseconds following the evoking stimulations.

20. The system of claim 18, wherein the evoked neural responses are indicative of synaptic activity within the patient's spinal cord.
